(19) United States
(12) Patent Application Publication
(10) Pub. No.: US 2023/0280338 A1
(43) Pub. Date: Sep. 7, 2023

(54) IMPROVEMENTS IN OR RELATING TO AN APPARATUS FOR DETECTION AND ANALYSIS OF A COMPONENT

(71) Applicant: VIDYA HOLDINGS LTD, London (GB)

(72) Inventors: Marko Dorrestijn, London (GB); David R. Klug, London (GB); Vladimir Turek, London (GB); Stefan Leo Van Workum, London (GB)

(73) Assignee: VIDYA HOLDINGS LTD, London (GB)

(21) Appl. No.: 18/015,463

(22) PCT Filed: Jul. 9, 2021
(86) PCT No.: PCT/GB2021/051771

§ 371 Date: Jan. 10, 2023

(30) Foreign Application Priority Data
Jul. 10, 2020 (GB) 2010690.2

(51) Int. Cl. G01N 33/543; G01N 21/63

(52) U.S. Cl. CPC G01N 33/54373; G01N 21/63; G01N 2201/121

(57) ABSTRACT

An apparatus for creating and correcting a two dimensional intensity map of one or more assay spots in a detection zone is provided. The apparatus comprising, a locator for affirming the location of the detection zone; a total internal reflection excitation device comprising a light source for illuminating the one or more assay spots in the detection zone; a detector configured to receive light that is emitted, reflected or scattered from the one or more assay spots and to create a two dimensional intensity map of the one or more assay spots comprising a two dimensional array of quantitative pixel values; and a processor configured to correct the intensity map to remove noise through analysis of corresponding pixel values from an earlier intensity map; wherein the analysis includes curve fitting.

The present invention relates to improvements in or relating to an apparatus for creating and correcting a two dimensional intensity map of one or more assay spots in a detection zone and, in particular, to an apparatus configured to correct the two dimensional intensity map to remove noise. In this context, a two dimensional map means monitoring the intensity of one or more locations in the detection zone.

Detecting a component such as a biomolecule has attracted great interest in early disease diagnosis, including cancers, inflammation and neurodegenerative diseases. The detection of components such as antibodies and other components of interest can facilitate early clinical or diagnostic treatment. There is growing interest in the development of ultrasensitive immunoassays to detect components of interest, especially at the single molecule sensitivity level.

Immunoassays can achieve up to single molecule sensitivity if configured appropriately. Single molecule sensitivity can be broadly achieved using two common strategies. The first strategy is to utilise high quality detection equipment such as cameras with objective lens, while the second strategy aims to increase the signal from the sample. Utilising high quality detection equipment can be very costly and labour intensive which often means that it can only be carried out in a laboratory setting. Moreover, methods for increasing the signal from the sample are often constrained by some material properties for example, fluorophore labels in immunoassays tend to give a relatively modest signal and therefore require sensitive detectors. By comparison, scattering labels, such as plasmonic nanoparticles (NP) can yield a signal that is five orders of magnitude greater than fluorophore labels. However, challenges still exist in differentiating signal from the scattering labels from the background signal.

The term background signal as used in the context of the present invention includes any change in the signal picked up by the detector either additional signals that do not arise from the assay or loss of signal caused by the absorption or obstruction of desired signal. Examples of background signals include, but are not limited to: detector noise; excess label species in the field of view; non-specific binding of the label to a surface, which may also include binding inside the capture region of the assay; autofluorescence; imperfections of the substrate during manufacture; scattering from large objects such as dust or from the sample and optical effects, for example, undesired reflections.

There are known methods for minimising the background signal during analysis of an assay sample for example; good optical design can minimise stray reflections or the use of total internal reflection (TIR) to minimise the signal from excess label. In addition, precision manufacturing processes of the substrate can also reduce substrate imperfection. However, it is not possible to completely eliminate the background signal especially when the field of view on the sample is quite large.

Therefore, there is a requirement to provide an apparatus and method to correct the background signal such that the background or noise signal is completely eliminated when detecting the analyte signal.

It is against this background that the invention has arisen.

According to the present invention there is provided an apparatus for creating and correcting a two dimensional intensity map of one or more assay spots in a detection zone, the apparatus comprising, a locator for affirming the location of the detection zone; a total internal reflection excitation device comprising a light source for illuminating the one or more assay spots in the detection zone; a detector configured to receive light that is emitted, reflected or scattered from the one or more assay spots and to create a two dimensional intensity map of the one or more assay spots comprising a two dimensional array of quantitative pixel values; and a processor configured to correct the intensity map to remove noise through analysis of corresponding pixel values from an earlier intensity map; wherein the analysis includes curve fitting.

In another aspect of the present invention, there is provided an apparatus for creating and correcting a two dimensional intensity map of one or more assay spots in a detection zone, the apparatus comprising, a locator for affirming the location of the detection zone; a total internal reflection excitation device comprising a light source for illuminating the one or more assay spots in the detection zone; a detector configured to receive light that is emitted, reflected or scattered from the one or more assay spots and to create a two dimensional intensity map of the one or more assay spots comprising a two dimensional array of quantitative pixel values; and a processor configured to correct the intensity map to remove noise through analysis of corresponding pixel values from an earlier intensity map.

The present invention as disclosed herein demonstrates how a series of two or more recordings on an assay that has not reached equilibrium, i.e. in cases where the label signal changes with time, enables subtraction of the background signal. This allows more reliable extraction of the rate of change of the assay strength over time and therefore provides an accurate measurement of the concentration of the target species of the assay.

In some embodiments, this processing of the data to remove the background on a pixel by pixel basis enables the signal from the assay spot or array of assay spots to be more easily discerned from the background. Depending on the configuration of the apparatus, the array of quantitative pixels could consist merely of two points which are selected to coincide with the centre of a single assay spot and a point beyond the assay spot thereby providing an intensity map differentiating a spot read out from readout away from a spot. Alternatively, the array of quantitative pixels may comprise an array of contiguous pixels, each providing data from a fraction of the area of the array. For example, there may be 256×256 pixels, or 1064×1064 pixels or 256×1064 pixels.

The term “noise” as disclosed in the present invention and within the context of this invention, should be understood to include any signal that is not arising from the specific binding events within the assay spots. Noise includes non-specific binding events involving the reagents in the detection zone; and noise from the background signal as defined above. In some embodiments, the assay spot may comprise one or more capture components and/or one or more detection reagents.

The capture component may be an antibody. Alternatively or additionally, the capture component could be a nucleic acid such as DNA, RNA, mRNA or microRNA, or chemically modified nucleic acid; it could be a protein, or a modified protein; or a peptide; or a polymer; it could be a hormone; or a tethered small molecule configured to capture a protein. In some embodiments, the capture component may be a non-specific capture component such as saliva or polylysine. The detection reagent, which can be a secondary antibody, and can be bound with a label can be disposed in various configurations.

Optionally, the label may be a fluorophore, a nanoparticle or a quantum dot. The label can be attached to the detection reagent.

The detection reagent can bind to the target component to form a detection reagent-target component complex. The detection reagent-target component complex can then bind to the capture component to form a sandwich complex. The detection reagent can either have inherent light emitting or scattering properties or the detection reagent may comprise a label with light emitting or scattering properties.

The detection reagents may be, but is not limited to, one or more of the following: a peptide, a protein, a protein assembly, an oligonucleotide, a polynucleotide, a modified oligonucleotide, a modified polynucleotide, an aptamer, a morpholino, a small molecule, a cell, a cell membrane, a viral particle, a glycan, a conjugated solid particle, a conjugated solid bead or a cofactor.

The label may be, but is not limited to, one or more of the following: a luminescence molecule; a fluorescent molecule; a phosphorescence molecule; a chemiluminescent molecule; a molecule that exhibits Rayleigh scattering or Raman scattering; a photon upconversion; an enzyme and its substrate that produces a colorimetric signal; a metallic or inorganic particles e.g. nanoparticles, a polycyclic aromatic hydrocarbon, a metalized complex, a quantum dot or an ion. The ion may be an atomistic ion or a salt of an organic molecule.

The label can be attached to the detection reagent.

Additionally or alternatively, the detection reagent may comprise an antibody. In some instances, the detection antibody can be fluorescently labelled.

The term “assay spot” within the context of the present invention is used to refer to a spot comprising a capture agent that captures an analyte of interest. The capture agent can form part of a competition assay or a sandwich assay.

In some embodiments, the analysis comprises subtraction of corresponding pixel values from an earlier intensity map.

In some embodiments, the analysis comprises identifying a step within a known range. This is applicable in scenarios where the detector frame rate exceeds the event rate. This ensures that each frame can only differ from the previous frame by a single event. The step in intensity provided by a specific binding event within the assay spot is known and therefore, if a step in intensity which corresponds to a specific binding event is identified, then it is validated as relevant data and processed as such. If a step in intensity occurs which does not correspond to the specific binding event value, then it must be noise and is discarded.

In some embodiments, the analysis may include curve fitting.

The term “curve fitting” as disclosed in the present invention and within the context of this invention, should be understood to include any analysis which establishes or quantifies kinetic behaviour or quantifies reaction dynamics. In some embodiments, curve fitting includes differentiation.

In some embodiments, the earlier corresponding intensity map can be detected at the time of manufacture of the detection zone and is provided to the apparatus. This can be particularly useful for taking into account features of the detection zone itself that would otherwise obscure the assay results.

In some embodiments, the processor can be configured to identify reference markers provided in or adjacent to the detection zone to align the earlier intensity map with the new intensity map.

This is important when the earlier intensity map is not prepared within the apparatus. For example, if the earlier intensity map is prepared as part of the manufacture of the detection zone.

In some embodiments, the earlier corresponding intensity map can be obtained in the apparatus. In situations where a series of intensity maps is obtained within the apparatus, there is no requirement for the reference markers because alignment is guaranteed as the detection zone is not movable with respect to the apparatus.

In some embodiments, the earlier intensity map can be obtained at T˜0 of the assay. This is the time when the assay is commenced and, depending on the exact configuration of the apparatus might be defined as the time when the apparatus was closed and the sample started to move towards the detection zone, or it might be the time when sample is first detected to be present in the detection zone and therefore the exact time at which the first capture components could form the sandwich assay with the detection reagent and target component complex. This earlier intensity map can be subtracted from each subsequent intensity map to remove background relating to bubbles, dust and other noise that remains unchanged throughout the assay.

In some embodiments, T˜0 may be defined by an actuation event which corresponds to the time at which the sample and detection reagent are brought into contact. The actuation event may be the closure of the device lid, the operation of a plunger located in the lid of the device, the operation of a pump, the opening of a valve within the device, the opening of a vent within the device, constriction or compression of a wall of the device, or the piercing of the lid or wall with a sharp object such as a syringe. In one embodiment, the opening of a valve or a vent downstream of the sample and detection reagent can be the actuation event.

In some embodiments, a plurality of intensity maps can be taken and each intensity map is corrected by subtraction of the directly preceding map. This can be referred to as continuous frame subtraction. Each intensity map is corrected using the n-1th intensity map. In this workflow it is critical that the time difference between each intensity map is sufficient for the assay to have progressed by a detectable amount.

In some embodiments, the processor can be further configured to process a plurality of corrected intensity maps to calculate the change of intensity over time of the one or more assay spots. This can provide a more robust data output because the change of intensity over time allows a direct determination of the change of concentration of the biomarker of interest over time thereby obviating the need to calibrate the apparatus and base the concentration data on a single reading. In some embodiments, the processor can be directly or indirectly coupled with the detector. In some embodiments the processor may be located in the cloud.

In some embodiments, the detection zone may be provided within a cartridge that is removable from the apparatus and wherein the locator is a support configured to hold the cartridge when it is inserted into the apparatus.

In some embodiments, the cartridge further comprises a unique identifier.

In some embodiments, the earlier corresponding intensity map can be detected at the time of manufacture of the cartridge and associated with the unique identifier.

The unique identifier may be provided as a QR code that may be read by the apparatus. The QR code may be associated with various unique data pertaining to the cartridge including the reagents included therein and the time of manufacture intensity map for that cartridge.

In some embodiments, the detection zone can be integral with the apparatus and the locator can be a reference marker to confirm the location of the detection zone.

In some embodiments, the total internal reflection excitation device may further comprises one or more lenses or mirrors in series configured to launch light towards the cartridge and to collect the emitted, reflected or scattered light from the one or more assay spots.

The provision of one or more lenses enables the total internal reflection excitation device to act as a microscope, magnifying the light emitted, reflected or scattered from the one or more assay spots. The microscope may be a total internal reflection microscope, or it may be a total internal reflection fluorescence (TIRF) microscope. TIRF microscopes can be used to detect emitted light derived from the assay spots. The detection of the emitted light can be, but is not limited to, fluorescence, luminescence or phosphorescence. The microscope may also be configured to detect Raman or Rayleigh scattering or photon upconversion molecules.

In some embodiments, the one or more assay spots may comprise a liquid sample. The liquid sample may be any bodily fluid including, but not limited to blood, serum, plasma, semen or saliva. In some embodiments, the liquid sample may be a saliva sample. Providing a saliva sample is a simple, non-intrusive procedure. As a result, users are typically more willing to provide a saliva sample than, for example, a blood sample.

The present invention will now be described, by way of example only, with reference to the accompanying figures in which:

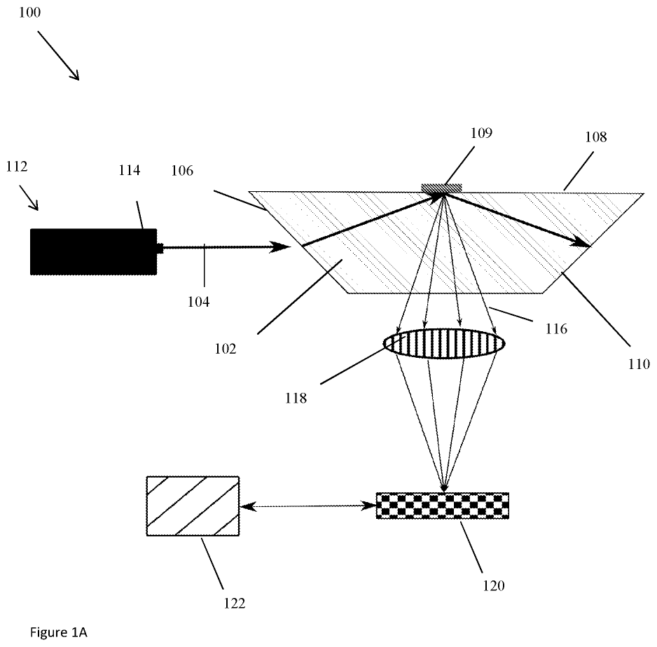

FIGS. 1A and 1B provide an apparatus according to the present invention;

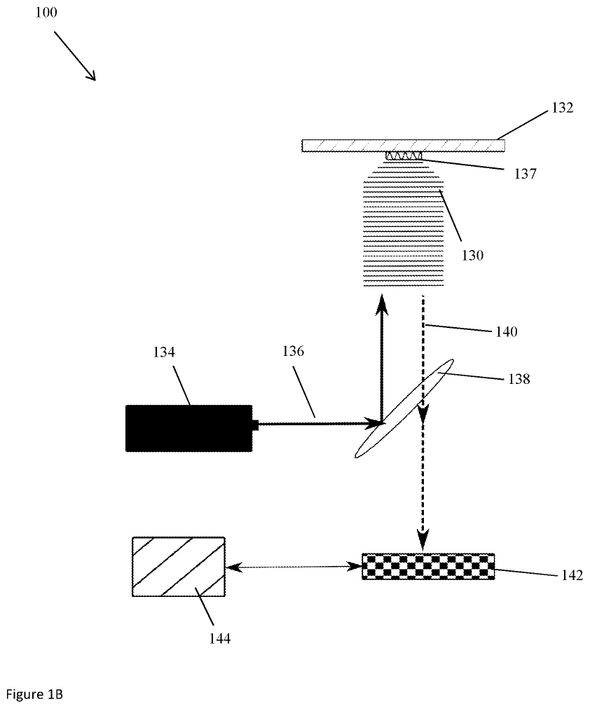

FIGS. 2A to 2C provide a schematic of the background subtraction according to the present invention;

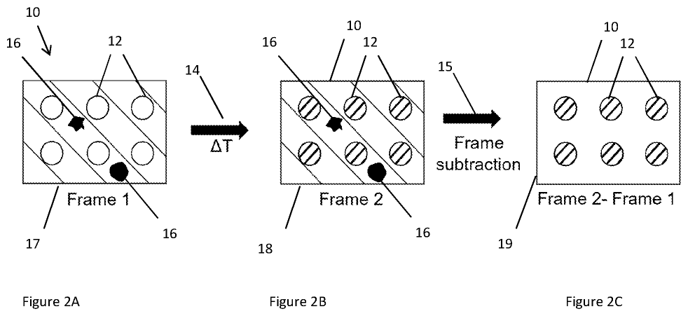

FIGS. 3A to 3D provide a schematic of a single frame subtraction;

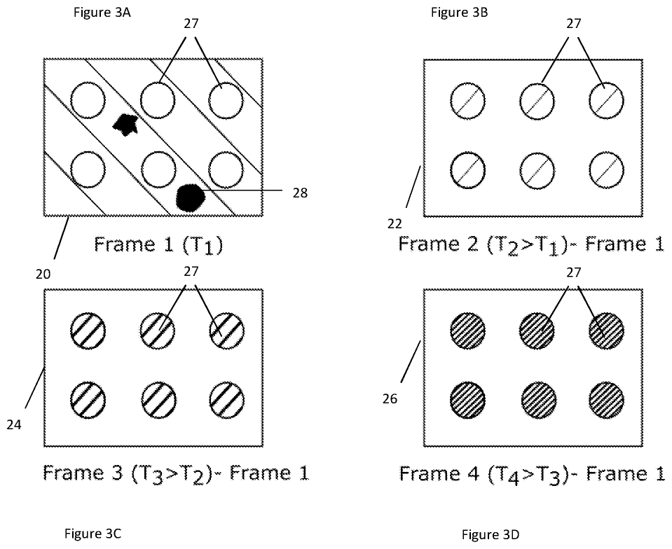

FIGS. 4A to 4D provide a schematic of the continuous frame subtraction method according to the present invention;

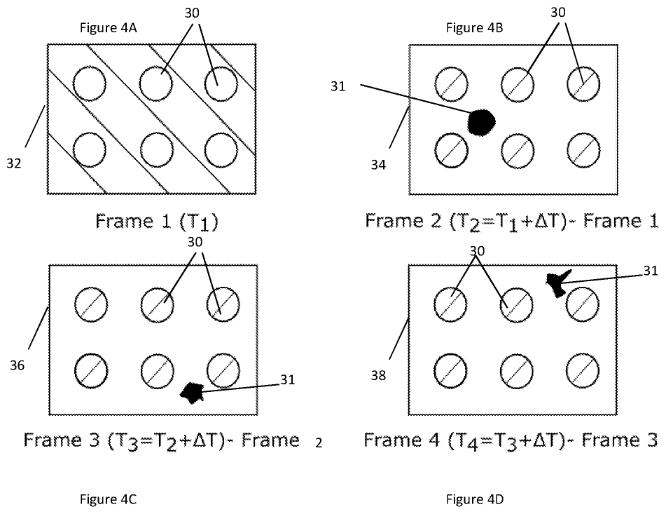

FIG. 5 provides a schematic for background subtraction in assays stored in a different medium to incubation;

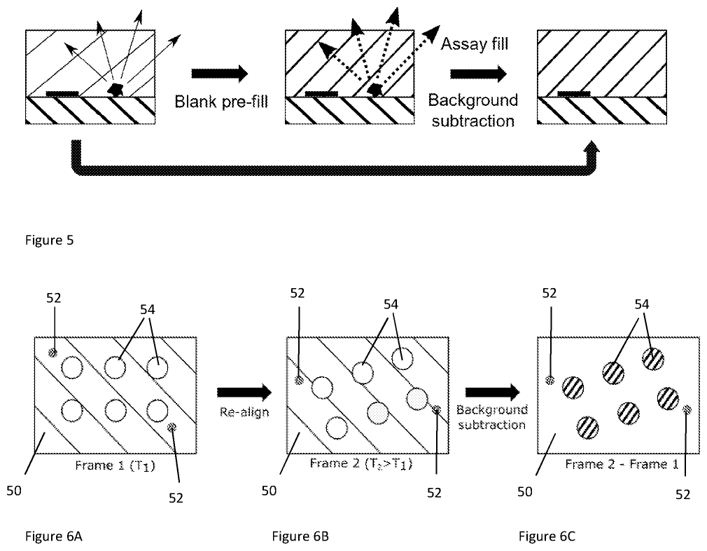

FIGS. 6A to 6C provide a schematic of background subtraction after realignment of the substrate;

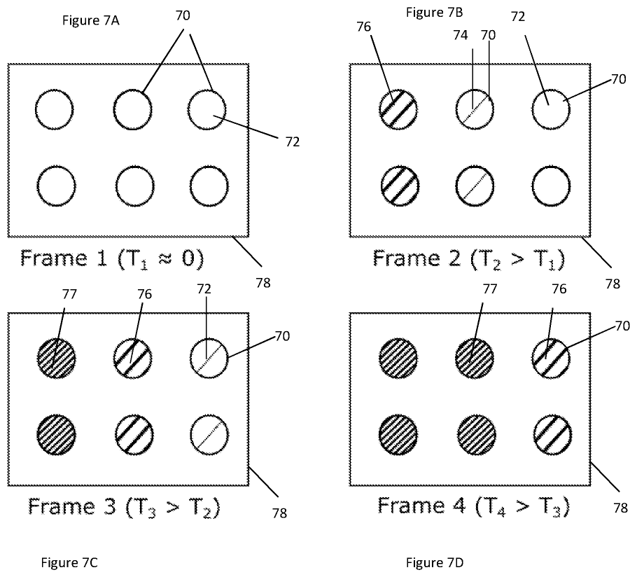

FIGS. 7A to 7D provide a schematic of kinetic multiplexing monitoring according to the present invention;

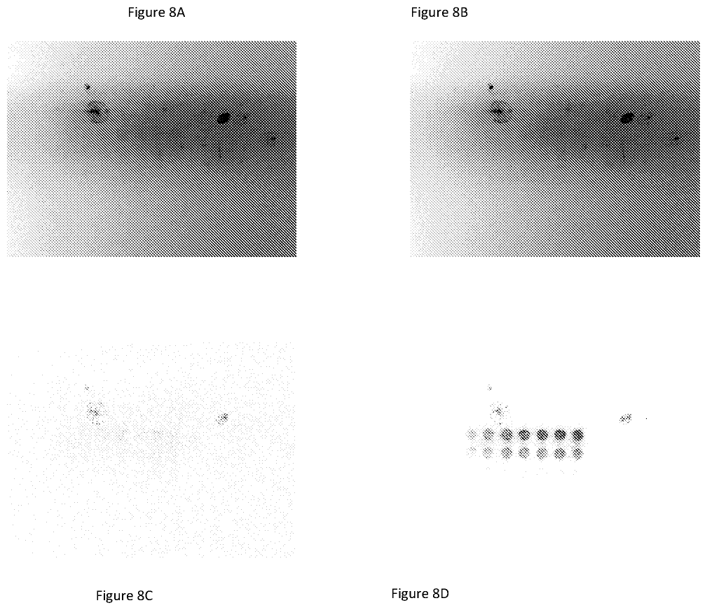

FIGS. 8A to 8D illustrate a subtraction method on a saliva sample;

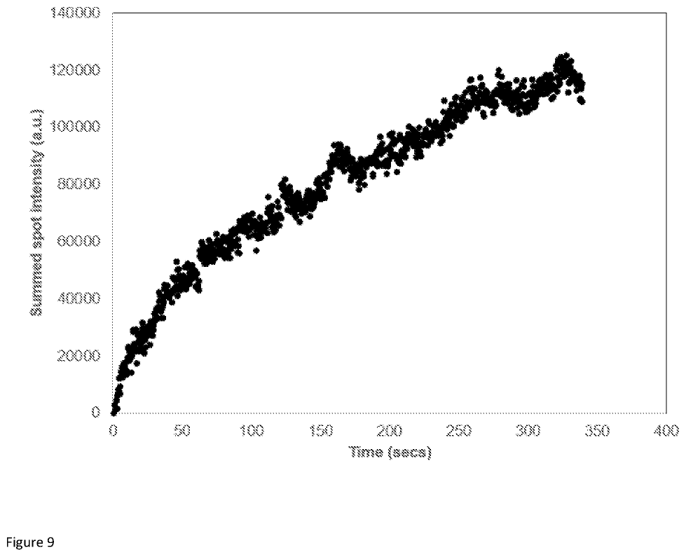

FIG. 9 shows a graph depicting assay intensity as a function of time;

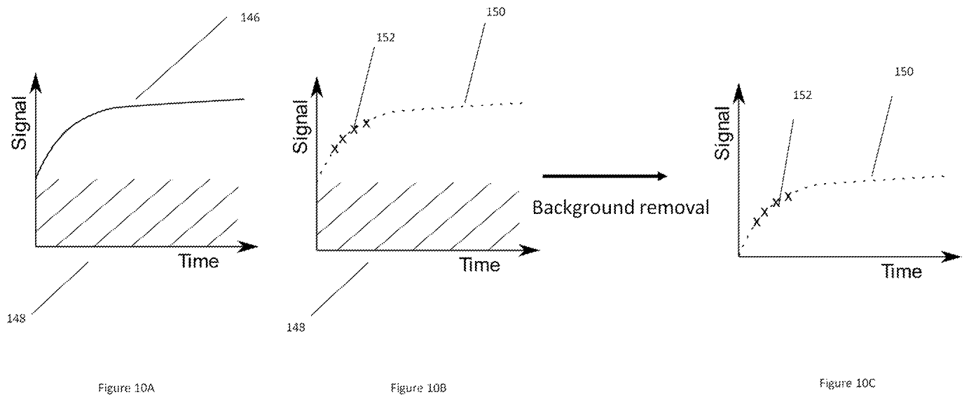

FIG. 10A shows a graph depicting assay signal as a function of time;

FIG. 10B shows a graph depicting a curve fit to measured assay points;

FIG. 10C shows a graph depicting assay signal as a function of time after a background subtraction; and

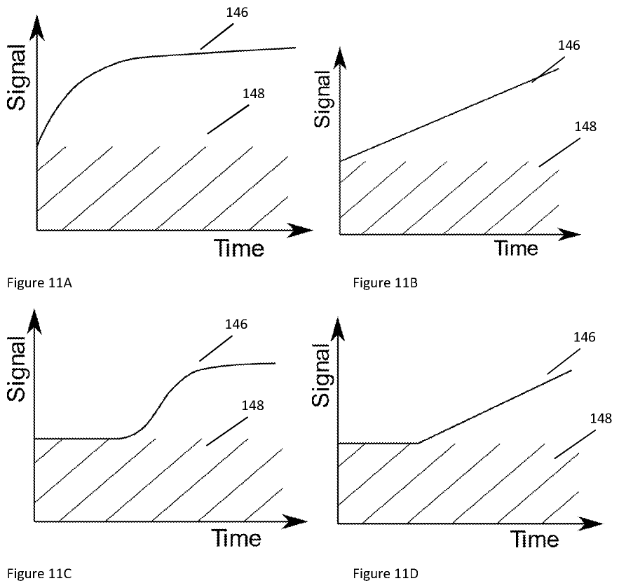

FIGS. 11A to 11D illustrate various graphs of assay signal as a function of time which may undergo curve fitting analysis.

The present invention as disclosed herein provides an apparatus and method for creating and correcting a two dimensional intensity map of one or more assay spots in a detection zone. Referring to FIG. 1A, there is provided an apparatus 100 for illustrating prism TIR geometry. The apparatus 100 comprises a substrate 102 such as an optical element 102 as shown in FIG. 1A. The optical element 102 comprises a light pathway 104 comprising an input surface 106, a reflective surface 108 and an output surface 110. The input surface 106 enables light 104, such as an incident light beam, to enter into the optical element 102. The input surface 106 and/or the output surface 110 can be a refractive or a diffractive or a transmissive surface. In some instances, the incident light beam is refracted at the input surface 106 upon entry into the optical element 102. The light 104 is directed towards the reflective surface 108 where one or more assay spots 109 comprising one or more capture components and/or one or more detection reagents are deposited onto the reflective surface 108 of the optical element 102. The assay spots are located in one or more detection zone on the surface of the optical element 102. In some instances, the incident light beam can be scattered or emitted upon entry into the optical element 102. The optical element 102 may be a prism as shown in FIG. 1A.

The apparatus 100 further comprises a total internal reflection excitation device 112 comprising a light source 114 for illuminating the one or more assay spots in the detection zone on the surface of the optical element 102. As shown in FIG. 1A, there is also provided a lens 118 such as an objective lens to collect the emitted, reflected or scattered light 116 at a specific wavelength from the assay spots deposited on the substrate 102. The lens 118 may be used to focus the emitted, reflected or scattered light 116 onto a detector 120. The detector 120 is configured to receive light that is emitted, reflected or scattered 116 from the one or more assay spots and to create a two dimensional intensity map of the one or more assay spots comprising a two dimensional array of quantitative pixel values. A processor 122, which can be directly or indirectly coupled with the detector 120, is configured to correct the intensity map to remove noise through analysis of corresponding pixel values from an earlier intensity map.

The apparatus 100 as disclosed herein involves acquiring more than one image of an immunoassay at various time points during an incubation period. By subtracting out pixel intensities between the frames, a ‘background removal’ method is attained. The residual between the subtracted frames is therefore the rate of change of the assay signal between the time points. This is in contrast with the traditional method of analysis whereby the signal of the assay is measured as a single acquisition and the intensity is calibrated through known standards. Kinetic measurements can be especially valuable in situations where the concentration of the component of interest or the label concentration varies with time or with respect to the absolute position of the field of view.

Referring to FIG. 1B, there is provided an apparatus 100 for illustrating objective TIR geometry. The apparatus 100 comprises an autofocus feedback system using a lens system 130. The lens system 130 may comprise one or more lenses arranged in series which can be configured to direct an incident light to a substrate 132 and to collect the emitted light, reflected or scattered light from the assay spots deposited on the substrate 132. The lens system 130 may comprise one or more objective lens. The apparatus 100 as shown in FIG. 1B comprises a substrate 132 where one or more assay spots are deposited onto the surface of the substrate 132. The assay spots are deposited, preferably printed onto the surface of the substrate 132. Each assay spot comprises at least one capture component and/or at least one detection reagent. A light source 134, for example a laser beam such as an exication laser beam, provides an incicident light 136 that can be directed at the substrate 132 to excite the molecules on the assay spots.

The apparatus 100 also comprises a refractive index matching liquid 137, which is provided between the lens system 130 and the substrate 132. Objective based TIR imaging requires refractive index matching liquid such as an index matching oil in between the assay spots deposited on the substrate and the lens system to achieve TIR. Additionally or alternatively, an index matching gel may be provided.

As shown in FIG. 1B, an exictation dichroic 138 is provided and configured to reflect the incident laser beam 136 into the lens system 130 and to reflect out the reflected, emitted or scattered light 140, as shown in FIG. 1B.

A detector 142 is then configured to receive the collected emitted, reflected or scattered light 140 from the lens system 130 to create a two dimensional intensity map of the one or more assay spots comprising a two dimensional array of quantitative pixel values. A processor 144, which can be directly or indirectly coupled with the detector 142, is configured to correct the intensity map to remove noise through analysis of corresponding pixel values from an earlier intensity map.

Referring to FIGS. 2A to 2C, there is provided an array 10 of assays spots 12 comprising one or more capture component and/or one or more detection reagents. The assay spots 12 that develop over time, as indicated by arrow 14, have a steady increase in intensity, whereas background contamination 16 occurs on different timescales or even remains unchanged over time. By subtracting out a prior frame, as indicated by arrow 15, such as subtracting frame 2 18 from frame 1 17, the subsequent images are cleaned from the background, as shown in frame 3 19 in FIG. 2C. By utilising this method, the detector which may be a camera, is able to detect the change in signal between different time points of the assay.

Referring to FIGS. 3A to 3D, there is a provided a single frame subtraction schematic. The recorded images 22, 24, 26 (FIGS. 3B to 3D) each have the initial recording 20 (see FIG. 3A) subtracted from them, yielding a background-free assay which increases with time. For assays that can be monitored continuously, the frame 20 when the incubation starts, or a short time after the start of the assay 27 as shown in FIG. 3A, can be used as the background level 28 and be removed from all subsequent frames 22, 24, 26 (FIGS. 3B to 2D). In some embodiments, it would be desirable and preferable for the subtracted frame as shown in FIG. 3D to be close to the start of the assay, because substantially all of the signal at that time will be background signal at T=0. This mode of assay tracking relies on the biggest difference between the images being the binding of the detection component of the assay for example, secondary antibodies to the capture component such as primary antibodies. The subtraction method as illustrated in FIGS. 3A to 3D is particularly useful when the profile of the laser illumination or the excess concentration of the label does not change significantly over time, which may occur from for example a moving liquid-air boundary due to evaporation or a changing concentration gradient of the detection reagents.

Referring to FIGS. 4A to 4D, there is provided an illustration of the continuous frame subtraction methodology. As illustrated in FIGS. 4A to 4D, frames 32, 34, 36, 38 are subtracted from each other after a pre-determined time. The background signal 31 that has developed during the assay 30 appears in only a single analysed frame.

The T=0 subtraction highlights the difference of the recorded image between the initial assay and all subsequent images. In some instances, the signal can in principle be subtracted from any time point. The signal subtraction can be particularly effective when the signal of the background 31 contamination is introduced prior to the assembly of the assay 30. If on the other hand the assay accumulates additional contamination during the incubation period, then T=0 subtraction will carry the additional background over. Given that the signal intensity of the assays follows approximately a linear relation at intermediate incubations, it is possible to continually subtract consecutive frames to afford a steady assay difference signal. Additional background during the incubation then contaminates only a single image before being removed again. Using this method, it can be critical to select a sufficient time between the subtracted frames to ensure that the assay intensity has changed substantially. During this time, the acquisition can either be inactive e.g. if the label is damaged by the acquisition, as is the case for fluorophores or the in-between frames can have further processing to produce a representative snapshot of the assay with the selected time window.

Referring to FIG. 5, a schematic is provided to show two methods for achieving a T<0 background subtraction in assays stored in a different medium to incubation. As shown in FIG. 5, the assay is filled and correlated for difference in intensity between the different media. The background signal is then subtracted.

Some detection reagents such as fluorophores can undergo photo-bleaching upon light exposure, which means that continuous acquisition would reduce the assay intensity and is therefore not feasible. Although photo-bleaching of a large number of molecules can be taken into the account, it may be beneficial to avoid unnecessary exposure of the assay. Provided that the background signal is introduced to the assay during manufacturing process and not from the assay itself, it may then be possible to record this before the start of the incubation. It may be a challenge in that the optical detection is sensitive to the medium in which the acquisition occurs. The assays are performed in a solution containing the sample and the detection reagents, whereas prior to the start of the incubation, the detection reagents are usually kept dry. The background of the dry state before the incubation cannot therefore be simply subtracted from the subsequent images when the incubation starts to remove the contaminant signal. This can be alleviated in two ways; the dry and the filled state is correlated and accounted for or the assay is pre-loaded with a liquid with similar optical properties to the sample/detection reagent mixture.

Referring to FIGS. 6A to 6C, there is shown a T<<0 subtraction schematic. After the initial background measurement as shown in FIG. 6A, the consumable 50 comprising one or more assay spots 54 needs to be re-aligned with respect to the reader (not shown in the accompanying drawings) as shown in FIG. 6B. In some instances where the consumable 50 is not perfectly aligned, the two reference spots 52 can help correlate both the positional and rotational mismatch to perform the background subtraction, as shown in FIG. 6C. The consumable 50 can be rotated at any angle such that it is correctly aligned with the reader.

To achieve a good quality T<0 background subtraction, it is desirable that the assay consumable 50 does not degrade or accumulate additional background. The subtraction can be done at any point prior to the assay. In some instances, the major source of background signal can be introduced during the manufacture of the consumable 50, then a T<<0 background subtraction can be performed as part of the quality control of the manufacture in batches, then stored until needed, thereby improving the quality of the assay and negating the need for a blank measurement when performing the assay. As an example, this can be required when performing a rapid test.

The following method as illustrated in FIGS. 6A to 6C requires an alignment process whereas the other methods as disclosed herein have relied on the sample being in the same location with respect to the reader. A T<<0 background subtraction requires the sample to be taken off the reader and the same location found when the assay is read. In some cases, it may be possible to align the sample such that there is a negligible positional difference between the initial background reading and the latter assay measurements. In some examples, the reference alignment marks or spots 52 can be used to provide the positional information of both stages of the reading. One of the most straightforward ways to provide the positional references is through two spots spaced as far from each other as possible, while still within the field of view of a single frame. There are numerous other reference markers that can be used in this way. Examples of markers may include, but is not limited to, writing a line on more than two spots.

As shown in FIG. 7, there is provided a schematic of kinetic monitoring of multiplexed assays. For example, one or more assay spots 70 may comprise one or more different capture component and/or one or more different detection reagent 72, 74, 76, 77 from another assay spot 70 in the same frame 78. Assays of different rate constant in the field of view can be tracked by recording multiple frames at intermediate time points for the assay. Alternatively, a single measurement when the assays have equilibrated may show a similar intensity.

When more than one assay 72, 74, 76, 77 is in the field of view of the camera at the same time, it may be beneficial to acquire the signals of different assays 72, 74, 76, 77 at different times. For example a very fast binding and/or high biomarker concentration assay may require measurements within minutes after the start of incubation, while a slow binding assay and/or low biomarker concentration assay may require measurement after a few hours. Acquiring multiple recordings of such assays simultaneously as shown in FIG. 7 means that any type of assays can be multiplexed together without the loss of information from the assays.

Referring to FIGS. 8A to 8D, there is provided an illustration of the T=0 subtraction. FIG. 8A shows T≈0 seconds after filling of the assay mixture. FIG. 8B shows the kinetic activity 2 seconds after frame A shown in FIG. 8A. FIG. 8C illustrates the residual intensity of the subtraction of frame A from frame B. FIG. 8D illustrates the residual intensity after T=0 subtraction after 2 minutes of incubation.

The intensity of the assay can be tracked either individually if the assay is multiplexed or as a whole. For example, FIG. 9 demonstrates a single spot as a function of time. The ability to track the assay intensity as a function of time rather than acquiring a single static image allows the direct determination of the biomarker concentration rather than relying on an equivalent assay conditions using calibration standards. Thus, tracking the assay intensity as a function of time provides a more robust method and opens the possibility of de-skilled use outside laboratory conditions.

Referring to FIGS. 10A, 10B and 10C, there are provided examples of curve fitting analysis removing noise from an intensity map. FIG. 10A depicts an example of a graph showing signal as a function of time. The graph has contributions from an assay signal 146, and also a background signal 148. The background signal 148 may remain constant throughout the assay, or may change at a different rate to the assay signal 146. As shown in FIG. 10B, actual signal measurements can be plotted as points 152 on a graph. The contribution of the background to these measured points 152 is unknown. By analysis of corresponding pixel values from an earlier intensity map, a curve 150 can be fit to the measured points 152. By fitting a curve 150 to the measured points 152, it is possible for the contribution of the background signal 148 to be subtracted, and for noise to be removed from the assay signal 146. Referring to FIG. 10C, after the background subtraction, the graph of signal as a function of time is free from background signal 148, which facilitates an accurate determination of the assay signal.

Referring to FIGS. 11A to 11D, there are provided examples of graphs of signal as a function of time. The graphs illustrate the different kinetics an assay signal 146 may have. Curve fitting analysis may be used to remove the background signal 148 from any of the various assay signals 146 shown. FIG. 11A shows an example of a logarithmic increase signal. As illustrated in FIG. 11A, the signal is observed when all assay components come into contact at t=0 and one or more of the components depletes within the timescale of the measurement.

FIG. 11B shows an example of a linear signal increase. As illustrated in FIG. 11B, the signal is observed when all assay components come into contact at t=0 and none of the components deplete substantially within the measurement time. In some instances, the signals may plateau when the assay has reached equilibrium.

FIG. 11C shows an example of a linear signal decrease. As illustrated in FIG. 11C, the signal is observed when all assay components come into contact at t=0, none of the components deplete within the measurement time and the signal decreases in the presence of the analyte of interest. The graph as shown in FIG. 11C i.e. a decreased signal vs time scenario, may be a particularly useful application for assays with a high off rate.

FIG. 11D shows an example of a logarithmic increase with a lag. As illustrated in FIG. 11D, the signal is observed when there is a lag between all assay components coming into contact and one or more of the components depletes within the timescale of the measurement.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1: An apparatus for creating and correcting an image of one or more assay spots in a detection zone prior to the assay reaching equilibrium, the apparatus comprising:
a substrate comprising one or more assay spots deposited thereon;
a reference marker for affirming the location of the detection zone;
a total internal reflection excitation device comprising a light source for illuminating the one or more assay spots in the detection zone;
a detector configured to receive light that is emitted, reflected or scattered from the one or more assay spots and to create an image of the one or more assay spots comprising a two dimensional array of quantitative pixel values; and
a processor configured to correct the image to remove noise by fitting a curve to the quantitative pixel values using corresponding pixel values from an earlier image,
wherein the processor is configured to identify reference markers provided in or adjacent to the detection zone to align the earlier image with the new image.

2: The apparatus according to claim 1, wherein the analysis comprises subtraction of corresponding pixel values from an earlier image.

3. (canceled)

4: The apparatus according to claim 1, wherein the earlier image is detected at the time of manufacture of the detection zone and is provided to the apparatus.

5. (canceled)

6: The apparatus according to claim 1, wherein the earlier image is obtained in the apparatus.

7: The apparatus according to claim 1, wherein the earlier image is obtained at T˜0 of the assay.

8: The apparatus according to claim 1, wherein a plurality of images is taken and each image is corrected by subtraction of the directly preceding image.

9: The apparatus according to claim 1, wherein the processor is further configured to process a plurality of corrected images to calculate the change of intensity over time of the one or more assay spots.

10: The apparatus according to claim 1, wherein the detection zone is provided within a cartridge that is removable from the apparatus and wherein the reference marker is a support configured to hold the cartridge when it is inserted into the apparatus.

11: The apparatus according to claim 10, wherein the cartridge further comprises a unique identifier.

12: The apparatus according to claim 1, wherein the earlier image is detected at the time of manufacture of the cartridge and associated with the unique identifier.

13: The apparatus according to claim 1, wherein the detection zone is integral with the apparatus and the reference marker is configured to confirm the location of the detection zone.

14: The apparatus according to claim 1, wherein the total internal reflection excitation device further comprises one or more lenses or mirrors in series configured to launch light towards the cartridge and to collect the emitted, reflected or scattered light from the one or more assay spots.

15: The apparatus according to claim 1, wherein the one or more assay spots comprise a liquid sample.

16: The apparatus according to claim 15, wherein the liquid sample is a saliva sample.
